(19) United States
(12) Patent Application Publication
(10) Pub. No.: US 2024/0081732 A1
(43) Pub. Date: Mar. 14, 2024

(54) METHOD OF DETECTING SLEEP DISORDER BASED ON EEG SIGNAL AND DEVICE OF THE SAME

(71) Applicant: National Taiwan University, Taipei (TW)

(72) Inventors: Phone LIN, Taipei (TW); XIN-XUE LIN, Taipei (TW)

(73) Assignee: National Taiwan University, Taipei (TW)

(21) Appl. No.: 18/465,927

(22) Filed: Sep. 12, 2023

(30) Foreign Application Priority Data
Sep. 14, 2022 (TW) 111134701

(51) Int. Cl. A61B 5/00; A61B 5/372

(52) U.S. Cl. CPC A61B 5/4815; A61B 5/372; A61B 5/4812; A61B 5/7267; A61B 5/7275

(57) ABSTRACT

The present invention discloses a method of detecting sleep disorder based on an EEG signal and device of the same. The method and device only need an EEG signal for analysis to determine sleep disorder and abnormal score. Therefore, the method and device may reduce cost of collecting physical information and avoid from uncomfortable feeling of user who wears several sensors.

### FIELD OF THE INVENTION

The present invention generally relates to a method of detecting sleep disorder and a device of the same. Specifically, the method of detecting sleep disorder and the device of the same are based on an electroencephalography (EEG) signal.

### BACKGROUND OF THE INVENTION

Traditionally, various kinds of physiological signals received from a great amount of sensors attaching to a body of a person are needed to identify sleeping disorder. The physiological signals may comprise electroencephalography (EEG) signal, electrocardiogram (ECG) signal, Electromyography (EMG) signal, electrooculogram (EOG) signal, airflow signal, respiration efforts signal, oxygen saturation signal, etc. A device having all of such sensors is quite expensive because the price of it is about 23 to 33 thousand dollars. Because a certain certification for operators is required, currently only medical institutions can use such a device. The person has to stay in a medical institution for a whole night to collect enough data. Therefore, it is needed for decreasing the number of the signals required for determining sleep disorder.

### SUMMARY OF THE INVENTION

An object of the present invention is to provide a method of detecting sleep disorder based on an electroencephalography (EEG) signal and a device of the same which generate a determination of detecting sleep and an anomaly score by analyzing one single signal, i.e. EEG signal, to reduce cost of collecting physiological signals and uncomfortable feeling of a user wearing a lot of sensors.

Another object of the present invention is to provide a method of detecting sleep disorder based on an EEG signal which identifies sleep disorder with three analyzing steps comprising collecting an EEG signal only and gets a sequence of sleep stage X⁽ⁱ⁾ through a feature extraction algorithm and a machine learning algorithm, assessing an anomaly score of the sequence of sleep stage X), and determining if the sequence of sleep stage X⁽ⁱ⁾ represents sleep disorder.

Another object of the present invention is to provide a method of detecting sleep disorder based on an EEG signal and a device of the same which provides an anomaly score which is highly-positive correlated with Apnea-Hypopnea Index (AHI), defined by experts of this domain, (correlation coefficient >0.7), verified with experiments. Therefore, the anomaly score is of considerable referential importance in identifying an extent of sleep disorder of a user.

Yet, another object of the present invention is to provide a method of detecting sleep disorder based on an EEG signal and a device of the same which, preferably, may be used at home and a sleep quality may be analyzed by a mobile application when transmitting monitored data received from an EEG sensor to a mobile phone via a wireless communication link, if supported by a medical equipment manufacturer. When the sequence of sleep stage X⁽ⁱ⁾ representing sleep disorder is detected, a user may be informed for seeking a formal diagnosis in a hospital.

An aspect of the present invention is to provide a method of detecting sleep disorder based on an EEG signal comprising dividing a EEG signal into sections, dividing a EEG signal into sections, classifying each section of the EEG signal into a plurality of sleep stages and determining that which sleep stage each section of the EEG signal is through a feature extraction algorithm and a machine learning algorithm so as to get a sequence of sleep stage X⁽ⁱ⁾; assessing an anomaly score of the sequence of sleep stage X⁽ⁱ⁾ with an anomaly detection technique for a discrete sequence; and determining if the sequence of sleep stage X⁽ⁱ⁾ represents sleep disorder with a predetermined threshold η, so that if a risk assessment function of anomaly V(X⁽ⁱ⁾, fᵣ, L) satisfies V(X⁽ⁱ⁾, fᵣ, L)>η, determining the sequence of sleep stage X⁽ⁱ⁾ represents sleep disorder, in which fᵣ(·) is a function determining a sleep pattern of sleep disorder, and L is a length of a sliding window.

An aspect of the present invention is to provide a device of detecting sleep disorder based on an EEG signal, comprising a communication unit and a programming unit. The communication unit receives a EEG signal from an EEG sensor. The programming unit is configured to divide a EEG signal into sections, classify each section of the EEG signal into a plurality of sleep stages and determining that which sleep stage each section of the EEG signal is through a feature extraction algorithm and a machine learning algorithm so as to get a sequence of sleep stage X⁽ⁱ⁾; assess an anomaly score of the sequence of sleep stage X⁽ⁱ⁾ with an anomaly detection technique for a discrete sequence; and determine if the sequence of sleep stage X⁽ⁱ⁾ represents sleep disorder with a predetermined threshold η, so that if a risk assessment function of anomaly V(X⁽ⁱ⁾, fᵣ, L) satisfies V(X⁽ⁱ⁾, fᵣ, L)>η, determine the sequence of sleep stage X⁽ⁱ⁾ represents sleep disorder, in which fᵣ(·) is a function determining a sleep pattern of sleep disorder, and L is a length of a sliding window.

### BRIEF DESCRIPTION OF THE DRAWINGS

Various objects and advantages of the present invention will be more readily understood from the following detailed description when read in conjunction with the appended drawing, in which:

FIG. 1 shows a sleep disorder detecting system according to an embodiment of the invention;

FIG. 2 illustrates a sleep disorder detecting method according to an embodiment of the invention;

FIG. 3 shows a perspective view of a waveform of an electroencephalography (EEG) signal according to an embodiment of the invention;

FIG. 4 shows a perspective view of a set of historical data Hₓ, as training data, and testing data according to an embodiment of the invention;

FIG. 5 shows a perspective view of a set of sliding window A_{L}(X) formed by a plurality of sliding windows, the length of which is L, taken out of the sequence of sleep stage X⁽ⁱ⁾, according to an embodiment of the invention;

FIG. 6 shows a perspective view of a set of all possible lookahead pairs is Bₗₒ(a) of a sleep pattern a according to an embodiment of the invention.

### DESCRIPTION OF EMBODIMENTS OF THE INVENTION

For a more complete understanding of the present disclosure and its advantages, reference is now made to the following description taken in conjunction with the accompanying drawings, in which like reference numbers indicate like features. Persons having ordinary skill in the art will understand other varieties for implementing example embodiments, including those described herein. The drawings are not limited to specific scale and similar reference numbers are used for representing similar elements. As used in the disclosures and the appended claims, the terms “embodiment,” “example embodiment,” and “present embodiment” do not necessarily refer to a single embodiment, although it may, and various example embodiments may be readily combined and interchanged, without departing from the scope or spirit of the present disclosure. Furthermore, the terminology as used herein is for the purpose of describing example embodiments only and is not intended to be a limitation of the disclosure. In this respect, as used herein, the term “in” may include “in” and “on”, and the terms “a”, “an” and “the” may include singular and plural references. Furthermore, as used herein, the term “by” may also mean “from”, depending on the context. Furthermore, as used herein, the term “if” may also mean “when” or “upon”, depending on the context. Furthermore, as used herein, the words “and/or” may refer to and encompass any and all possible combinations of one or more of the associated listed items.

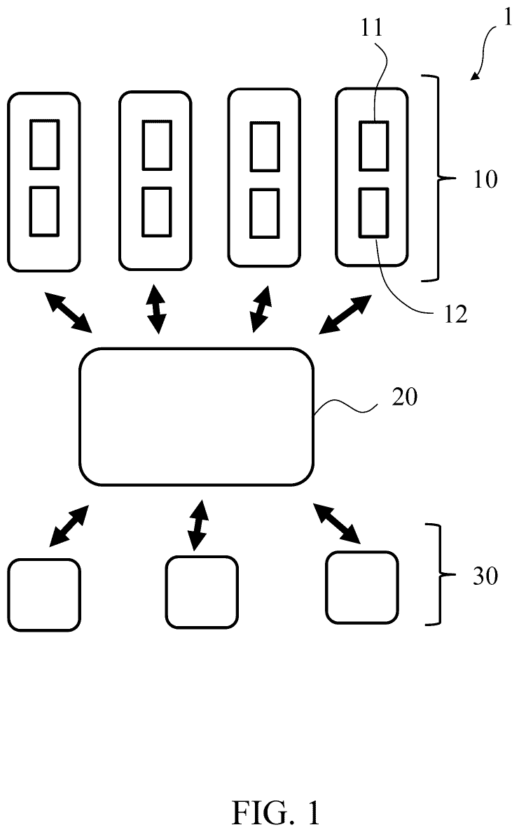

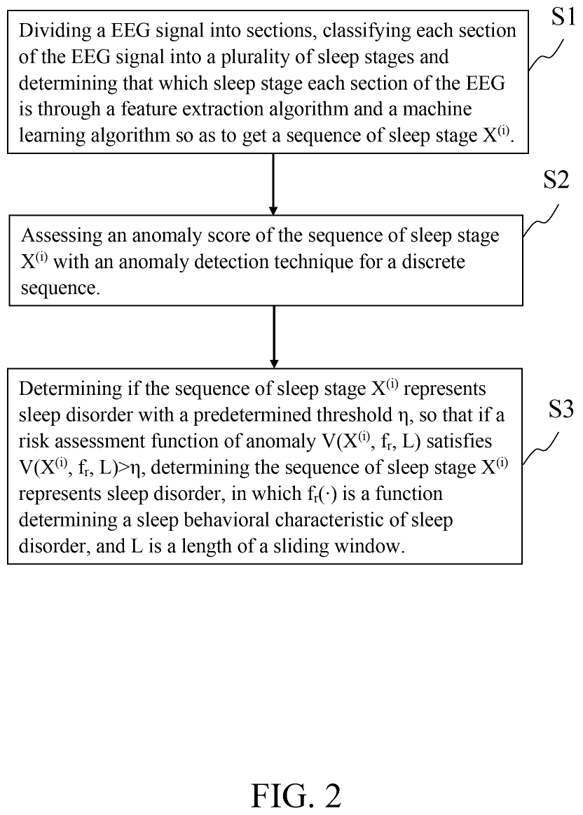

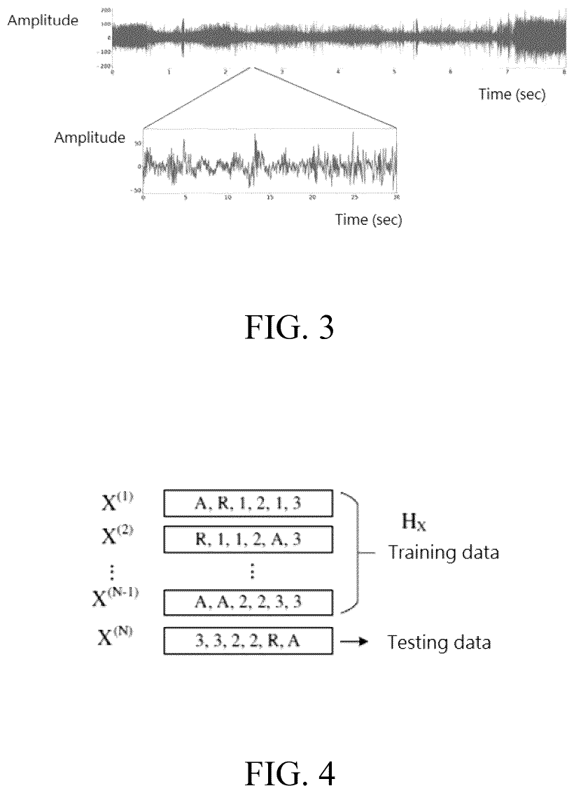

In the present specification, several examples of a method of detecting sleep disorder based on an electroencephalography (EEG) signal and a device of the same are disclosed. Please refer to FIGS. 1 to 3, in which FIG. 1 shows an sleep disorder detecting system 1 according to an embodiment of the invention, comprising a plurality of sleep disorder detecting devices 10, a communication platform 20 and a plurality of EEG sensors 30. The sleep disorder detecting devices 10 are adapted to apply an embodiment of a method of detecting sleep disorder, such as the one shown in FIG. 2, showing a sleep disorder detecting method according to an embodiment of the invention, comprising steps S1, S2 and S3. FIG. 3 shows a perspective view of a waveform of an EEG signal according to an embodiment of the invention. Please note that the sleep disorder detecting devices 10 are merely an exemplary embodiment of various types of sleep disorder detecting device implementing the method of detecting sleep disorder, so that the method of detecting sleep disorder is not tied to the example disclosed here. The sleep disorder detecting devices 10 may comprise a communication unit 11 and a programming unit 12. The communication platform 20 may form communication links in complying with at least one communication protocol with the communication unit 11 of the sleep disorder detecting devices 10 and the EEG sensors 30, so that the communication unit 11 may receive an EEG signal from the EEG sensors 30. The communication protocol may be but not limited to a wireless communication protocol, such as Bluetooth wireless communication protocol, Wi-Fi wireless communication protocol, etc., and the communication unit 11 may be corresponding Bluetooth wireless communication unit, Wi-Fi wireless communication unit, etc. In the present embodiment, the sleep disorder detecting devices 10 may be mobile phones, which are adapt to cooperate with the sleep disorder detecting system 1, constructed by a medical equipment manufacturer, that transmits data monitored by the EEG sensors 30 to the mobile phones via the one of the wireless communication link, such as Bluetooth, Wi-Fi communication link, etc. for a mobile application to analyze a sleep quality. If an anomaly is detected, a user is informed for formal diagnosis in a hospital.

The programming unit 12, which may be implemented as a processor, microprocessor, central processing unit, etc., may be configured to execute steps S1, S2 and S3 of the method of detecting sleep disorder. After the sleep disorder detecting devices 10 receive the EEG signal, in the step S1, the programming unit 12 may divide a EEG signal into sections, classify each section of the EEG signal into a plurality of sleep stages and determining that which sleep stage each section of the EEG signal is through a feature extraction algorithm and a machine learning algorithm so as to get a sequence of sleep stage X). Please note that, compared with conventional methods, the method of detecting sleep disorder according to the present invention only analyzes one single EEG signal to determine if sleep disorder exists and an anomaly score, so as to reduce cost of collecting physiological signals and uncomfortable feeling of the user wearing a lot of sensors. The programming unit 12 may divide the EEG signal from the EEG sensors 30 into sections of a fixed length, and preferably, the fixed length may be between 10 seconds and 1 minute. For the example shown in FIG. 3, an upper half shows the EEG signal from the EEG sensors 30, and an lower half shows an example of a section of the EEG signal a length of which is 30 seconds. Here, the programming unit 12 may classify each of the sections of EEG signal into several sleep stages, such as five standard sleep stages: awake stage, REM stage, N1 stage, N2 stage and N3 stage. Then, the programming unit 12 may determine that which sleep stage each section of the EEG signal is through a feature extraction algorithm and a machine learning algorithm. Here, the machine learning algorithm may be but not limited to convolutional neural network (CNN), recurrent neural network (RNN), random forests, etc., and the feature extraction algorithm may be but not limited to Fourier transform, wavelet analysis, short-time Fourier transform (STFT), autoregressive model, etc. After performing the feature extraction algorithm and the machine learning algorithm, the sequence of sleep stage X⁽ⁱ⁾ is formed.

Then, in the step S2, the programming unit 12 may assess an anomaly score of the sequence of sleep stage X⁽ⁱ⁾ with an anomaly detection technique for a discrete sequence. When it is assumed that the sequence of sleep stage X⁽ⁱ⁾ to be assessed is set as X⁽ⁿ⁾=(X⁽ⁿ⁾₍₁₎, X⁽ⁿ⁾₍₂₎, X⁽ⁿ⁾₍₃₎, . . . , X⁽ⁿ⁾₍ₘ₎), in which X⁽ⁱ⁾₍ⱼ₎ belongs to a set of {A, R, 1, 2,3} and A, R, 1, 2,3 correspond to the five sleep stages, i.e. awake stage, REM stage, N1 stage, N2 stage and N3 stage, respectively, the programming unit 12 may take a plurality of sliding windows, the length of which is L, out of the sequence of sleep stage X⁽ⁱ⁾ as sleep patterns of sleep for each historical data in a set of historical data Hₓ={X⁽ⁱ⁾, X⁽²⁾, . . . , X⁽ⁿ⁻¹⁾} to form a set of sliding window A_{L}(X), in which a set of all sliding windows in the historical data is H_{A} which satisfies H_{A}=∪ {A_{L}(h)|h ∈Hₓ}=A_{L}(X⁽¹⁾)∪ A_{L}(X⁽²⁾)∪ . . . ∪ A_{L}(X⁽ⁿ⁻¹⁾). The historical data Hₓ may be used as training data, as shown in FIG. 4.

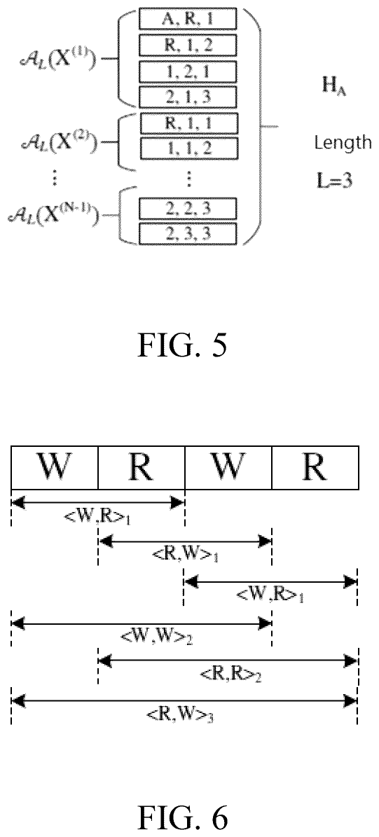

The programming unit 12 may then form the sliding window for each historical data with the length L. A_{L}(X) represents the set of all the sliding window, the length of which is L, taken from the sequence of sleep stage X. As an example shown in FIG. 5, L=3, X⁽¹⁾=(A,R,1,2,1,3), and the sliding windows, the length of which is 3, are (A,R,1), (R,1,2), (1,2,1) and (2,1,3). The set formed by these sliding windows is represented by A₃(X⁽¹⁾) here.

For a sliding window, or a so-called sleep pattern, a lookahead pair may be defined and represented by <x,y>ᵢ. The programming unit 12 may define a lookahead pair <x,y>ᵢ of a sleep pattern a=(a₁, a₂, . . . , a_{L}) as a subsequence (aₘ, aₙ) of a, in which m, n, i are integers, 1≤m,n,i≤L, x=aₘ, y=aₙ and n−m=i. Further, a set of all possible lookahead pairs is Bₗₒ(a), which satisfies Bₗₒ(a)={<aₘ, aₙ>ₖ|∃m,n,k∈ [symbol] s.t. 1≤m,n,k≤L and k=m−n}. As shown in the example in FIG. 6, the set of all possible lookahead pairs Bₗₒ(a) is {<W,R>₁, <R,W>₁, <W,W>₂, <R,R>₂, <R,W>₃} of a sleep pattern a=(W,R,W,R).

The programming unit 12 then may set <x,y>ᵢ as a lookahead pair, and use C(<x,y>ᵢ, H_{A}) to represent a number of <x,y>ᵢ shown in the set H_{A}, and define that C(<x,y>ᵢ, H_{A})=|{a|a∈ H_{A} and <x,y>ᵢ ∈ Bₗₒ(a)}|, in which |·| represents an element number of a set. The programming unit 12 may also define a function determining a sleep pattern of sleep disorder fᵣ(·), an input of which is a sleep pattern a, as fᵣ(a)=1 if |{z|z∈Bₗₒ(a) and C(z, H_{A})/|H_{A}|<θ}|>0, and fᵣ(a)=0 if |{z|z∈Bₗₒ(a) and C(z, H_{A})/|H_{A}|<θ}|=0, in which θ is another predetermined threshold. A risk assessment function of anomaly V(X⁽ⁱ⁾, fᵣ, L) may be defined as V(X⁽ⁱ⁾, fᵣ, L)=(sum {fᵣ(a)|a∈A_{L}(X⁽ⁱ⁾})/(|X⁽ⁱ⁾|+L−1), in which 0≤V(X⁽ⁱ⁾, fᵣ, L)≤1. Afterwards, the programming unit 12 may calculate an abnormal score of the sequence of sleep stage X⁽ⁱ⁾ with the risk assessment function of anomaly V(X⁽ⁱ⁾, fᵣ, L). Here, the abnormal score of the sequence of sleep stage X⁽ⁱ⁾ may represent an extent of sleep disorder, and the higher the abnormal score of the sequence of sleep stage X⁽ⁱ⁾ is, the greater the an extent of sleep disorder is, for example. Verified with experiments, it is found that the abnormal score defined here is highly-positive correlated with Apnea-Hypopnea Index (AHI) (correlation coefficient >0.7). Therefore, the anomaly score is of considerable referential importance in identifying an extent of sleep disorder of the user.

Then, in the step S3, the programming unit 12 may determine if the sequence of sleep stage X⁽ⁱ⁾ represents sleep disorder with a predetermined threshold η. Here, if the risk assessment function of anomaly V(X⁽ⁱ⁾, fᵣ, L) satisfies V(X⁽ⁱ⁾, fᵣ, L)>η, it is determined that the sequence of sleep stage X⁽ⁱ⁾ represents sleep disorder. The predetermined threshold η may be but not limited to set to meet actual requirement.

As mentioned above, the method of detecting sleep disorder based on the EEG signal and the device of the same of the present invention only collect one single EEG signal, and detect sleep disorder by performing three analysis steps with the collected EEG signal: getting the sequence of sleep stage X⁽ⁱ⁾ with the feature extraction algorithm and the machine learning algorithm, assessing the anomaly score of the sequence of sleep stage X⁽ⁱ⁾ and determining if the sequence of sleep stage X⁽ⁱ⁾ represents sleep disorder.

It is to be understood that these embodiments are not meant as limitations of the invention but merely exemplary descriptions of the invention with regard to certain specific embodiments. Indeed, different adaptations may be apparent to those skilled in the art without departing from the scope of the annexed claims.

What is claimed is:

1. A method of detecting sleep disorder based on electroencephalography (EEG) signal, comprising steps of:
dividing an EEG signal into sections, classifying each section of the EEG signal into a plurality of sleep stages and determining that which sleep stage each section of the EEG signal is through a feature extraction algorithm and a machine learning algorithm so as to get a sequence of sleep stage X⁽ⁱ⁾;
assessing an anomaly score of the sequence of sleep stage X⁽ⁱ⁾ with an anomaly detection technique for a discrete sequence; and
determining if the sequence of sleep stage X⁽ⁱ⁾ represents sleep disorder with a predetermined threshold η, so that if a risk assessment function of anomaly V(X⁽ⁱ⁾, fᵣ, L) satisfies V(X⁽ⁱ⁾, fᵣ, L)>η, determining the sequence of sleep stage X⁽ⁱ⁾ represents sleep disorder, in which fᵣ(·) is a function determining a sleep pattern of sleep disorder, and L is a length of a sliding window.

2. The method of detecting sleep disorder based on EEG signal according to claim 1, wherein the step of dividing a EEG signal into sections, classifying each section of the EEG signal into a plurality of sleep stages and determining that which sleep stage each section of the EEG signal is through a feature extraction algorithm and a machine learning algorithm so as to get a sequence of sleep stage X⁽ⁱ⁾ further comprises:
classifying each section of the EEG signal into a plurality of standard sleep stages which comprise awake stage, REM stage, N1 stage, N2 stage and N3 stage.

3. The method of detecting sleep disorder based on EEG signal according to claim 1, wherein the EEG signal is divided into sections of a fixed length which is between 10 seconds and 1 minute.

4. The method of detecting sleep disorder based on EEG signal according to claim 1, wherein the machine learning algorithm comprises one of convolutional neural network (CNN), recurrent neural network (RNN) and random forests.

5. The method of detecting sleep disorder based on EEG signal according to claim 1, wherein the feature extraction algorithm comprises one of Fourier transform, wavelet transform, short-time Fourier transform and autoregressive model extracting a feature.

6. The method of detecting sleep disorder based on EEG signal according to claim 1, wherein the step of assessing an anomaly score of the sequence of sleep stage X⁽ⁱ⁾ with an anomaly detection technique for a discrete sequence further comprises:
setting the sequence of sleep stage X⁽ⁱ⁾ as X⁽ⁿ⁾=(X⁽ⁿ⁾₍₁₎, X⁽ⁿ⁾₍₂₎, X⁽ⁿ⁾₍₃₎, . . . , X⁽ⁿ⁾₍ₘ₎), in which X⁽ⁱ⁾₍ⱼ₎ belongs to a set of {A, R, 1, 2,3} and A, R, 1, 2,3 correspond to five sleep stages respectively; and
taking a plurality of sliding windows, the length of which is L, out of the sequence of sleep stage X⁽ⁱ⁾ as sleep patterns of sleep for each historical data in a set of historical data Hₓ={X⁽¹⁾, X⁽²⁾, . . . , X⁽ⁿ⁻¹⁾} to form a set of sliding window A_{L}(X), in which a set of all sliding windows in the historical data is H_{A} which satisfies H_{A}=∪ {A_{L}(h)|h ∈Hₓ}=A_{L}(X⁽¹⁾) ∪ A_{L}(X⁽²⁾) ∪ . . . ∪ A_{L}(X⁽ⁿ⁻¹⁾).

7. The method of detecting sleep disorder based on EEG signal according to claim 6, wherein the step of assessing an anomaly score of the sequence of sleep stage X⁽ⁱ⁾ with an anomaly detection technique for a discrete sequence further comprises:
defining a lookahead pair of a sleep pattern of sleep in the set H_{A} is <x,y>_{I}, in which the sleep pattern a is a=(a₁, a₂, . . . , a_{L}), the lookahead pair is a subsequence of a and represented by (aₘ, aₙ), m, n, i are positive integers, aₘ, aₙ, m, n, i satisfy 1≤m, n, i≤L, x=aₘ, y=aₙ and n-m=i; and
defining a set of all possible lookahead pairs is Bₗₒ(a)={<aₘ, aₙ>ₖ| ∃m,n,kE [symbol] s.t. 1≤m,n,k≤L and k=m−n};
defining C(<x,y>ᵢ, H_{A})=|{a|a∈ H_{A} and <x,y>ᵢ ∈ Bₗₒ(a)}|, in which <x,y>ᵢ is a lookahead pair, C(<x,y>ᵢ, H_{A}) represents a number of <x,y>ᵢ in the set H_{A}, and |·| represents an element number of a set;
defining the function determining a sleep pattern of sleep disorder fᵣ(·), an input of which is a sleep pattern a, as fᵣ(a)=1 if |{z|z∈Bₗₒ(a) and C(z, H_{A})/|H_{A}|<θ}|>0, and fᵣ(a)=0 if |{z|z∈Bₗₒ(a) and C(z, H_{A})/|H_{A}|<θ}|=0, in which θ is another predetermined threshold;
defining the risk assessment function of anomaly V(X⁽ⁱ⁾, fᵣ, L) as V(X⁽ⁱ⁾, fᵣ,
L)=(sum{fᵣ(a)|a∈A_{L}(X⁽ⁱ⁾)})/(|X⁽ⁱ⁾)|+L−1), 0≤V(X⁽ⁱ⁾, fᵣ, L)≤1; and
calculating the abnormal score of the sequence of sleep stage X⁽ⁱ⁾ with the risk assessment function of anomaly V(X⁽ⁱ⁾, fᵣ, L).

8. The method of detecting sleep disorder based on EEG signal according to claim 6, wherein the abnormal score of the sequence of sleep stage X⁽ⁱ⁾ represents an extent of sleep disorder, and the higher the abnormal score of the sequence of sleep stage X⁽ⁱ⁾ is, the greater the an extent of sleep disorder is.

9. A device of detecting sleep disorder based on electroencephalography (EEG) signal, comprising:
a communication unit, receiving an EEG signal from an EEG sensor; and
a programming unit, configured to:
divide a EEG signal into sections, classify each section of the EEG signal into a plurality of sleep stages and determining that which sleep stage each section of the EEG signal is through a feature extraction algorithm and a machine learning algorithm so as to get a sequence of sleep stage X⁽ⁱ⁾;
assess an anomaly score of the sequence of sleep stage X⁽ⁱ⁾ with an anomaly detection technique for a discrete sequence; and
determine if the sequence of sleep stage X⁽ⁱ⁾ represents sleep disorder with a predetermined threshold η, so that if a risk assessment function of anomaly V(X⁽ⁱ⁾, fᵣ, L) satisfies V(X⁽ⁱ⁾, fᵣ, L)>η, determine the sequence of sleep stage X⁽ⁱ⁾ represents sleep disorder, in which fᵣ(·) is a function determining a sleep pattern of sleep disorder, and L is a length of a sliding window.

10. The device of detecting sleep disorder based on EEG signal according to claim 9, wherein the device is a mobile phone, the communication unit of which is one of a Bluetooth wireless communication unit and a Wi-Fi wireless communication unit.
